(19) United States
(12) Patent Application Publication
(10) Pub. No.: US 2002/0122828 A1
(43) Pub. Date: Sep. 5, 2002

(54) Hybrid porous materials for controlled release

(76) Inventor: Jun Liu, Murray Hill, NJ (US)

Correspondence Address: Douglas E McKinley Jr, P O Box 202, Richland, WA, 99352, US

(21) Appl. No.: 09/798,603

(22) Filed: Mar. 2, 2001

(51) Int. Cl. A61K 9/14; A61K 9/16; A61K 9/50

(52) U.S. Cl. 424/497

(57) ABSTRACT

Hybrid porous materials useful for releasing bioactive materials in response to an external stimulus at a constant release rate are formed of a porous inorganic gel having a polymer network integrated within the pores of a porous gel. The hybrid porous material is made by forming a mixture of a polymer precursor, a cross linking agent, an initiator, an alcohol, a ceramic precursor, water and an acid. The mixture is then formed either by first polymerizing the polymer precursor to form a polymer network, and then forming the ceramic precursor into a porous inorganic gel, or by first forming the ceramic precursor into a porous inorganic gel and then polymerizing the polymer precursor to form a polymer network. Either approach will yield a porous inorganic gel having a polymer network integrated within the pores of the porous gel.

This invention was made with Government support under Contract DE-AC0676RLO1830 awarded by the U.S. Department of Energy. The Government has certain rights in the invention.

### FIELD OF THE INVENTION

The present invention is a hybrid porous material for controlled release of a bioactive material. More specifically, the present invention is a nanoporous or microporous inorganic material with an interpenetrating network of an environmentally sensitive polymer.

### BACKGROUND OF THE INVENTION

A wide variety of materials have been investigated to provide for the controlled release of biologically active materials, such as pharmaceuticals or pesticides. For example, in U.S. Pat. No. 5,702,716 “Polymeric compositions useful as controlled release implants” Dunn et al. describe a combination of a thermoplastic polymer, a rate modifying agent, a bioactive material and an organic solvent. As described by Dunn et al., the liquid composition is capable fo forming a biodegradable and/or bioerodible microporous, solid matrix useful as an implant in patients (human and animal) for the delivery of bioactive substances to tissues or organs.

U.S. Pat. Nos. 4,474,751; 4,474,752; 4,474,753 generally describe the application of selected polymers as a novel drug delivery system which uses the body temperature and pH to induce a liquid to gel transition of the polymer which contains a drug or therapeutic agent therein. Similarly, U.S. Pat. No. 4,478,822 relates to a drug delivery system for delivering drugs to a body cavity. The drug delivery system comprises a medicament and a polymer such that the drug delivery system is a liquid at room temperature but forms a semi-solid or gel at the body temperature in the body cavity.

U.S. Pat. No. 4,895,724 describes compositions for the controlled and prolonged release of macromolecular compounds comprising a porous matrix of chitosan having dispersed therein the macromolecular compound. Examples of macromolecules used in the composition are pharmacologically active ones such as peptide hormones, e.g. growth hormone.

U.S. Pat. No. 4,833,660 describes gel bases for pharmaceutical compositions comprising from about 0.5 to about 10.0% by weight ethoxylated (2 to 30 moles of ethoxylation) behenyl alcohol and from about 90 to 99.5% of a glycol solvent or from about 2.5 to about 10.0% by weight ethoxylated fatty alcohols having a chain length of from 16 to 21 carbon atoms and from 90 to about 97.5% of a glycol solvent. Preferred glycol solvents include propylene glycol and polyethylent glycols having an average molecular weight of about 200 to 800. Pharmaceutical compositions suitable for topical, transmucosal and oral administration are prepared utilizing the gel bases. Methods of administration of topically, systemically and orally active pharmaceutical agents utilizing the gel bases are also described.

U.S. Pat. No. 4,861,760 describes pharmaceutical compositions intended for contacting with a physiological liquid characterized in that the composition is intended to be administered as a non-gelled liquid form and is intended to gel in situ. The composition contains at least one polysaccharide in aqueous solution, of the type which undergoes liquid-gel phase transition gelling in situ under the effect of an increase in the ionic strength of said physiological liquid.

U.S. Pat. No. 4,795,642 describes a controlled-release pharmaceutical unit dosage form provided as comprising a gelatin capsule enclosing a solid matrix formed by the cation-assisted gellation of a liquid fill incorporating vegatable gum and a pharmaceutically-active compound, as well as methods for the preparation thereof.

A disadvantage of these and other known systems is related to the relatively rapid release rate of the bioactive materials from the various polymer systems used to contain the bioactive materials.

Thus, there remains a need for systems providing a continuous release of biologically active materials, such as pharmaceuticals or pesticides.

### SUMMARY OF THE INVENTION

Accordingly, it is an object of the present invention to provide a hybrid porous material useful for releasing bioactive materials in response to an external stimulus at a constant release rate. It is a further object of the present invention to provide a method for making a hybrid porous material useful for releasing bioactive materials in response to an external stimulus at a constant release rate. These and other objects of the present invention are accomplished by the method for forming a hybrid porous material for controlled release of a bioactive material, and the hybrid porous material formed thereby described herein.

The hybrid porous material is made by forming a mixture of a polymer precursor, a cross linking agent, an initiator, an alcohol, a ceramic precursor, water and an acid. The mixture is then formed either by first polymerizing the polymer precursor to form a polymer network, and then forming the ceramic precursor into a porous inorganic gel, or by first forming the ceramic precursor into a porous inorganic gel and then polymerizing the polymer precursor to form a polymer network. Either approach will yield a porous inorganic gel having a polymer network integrated within the pores of the porous gel.

Polymerizing the polymer precursor is accomplished by exposing the polymer precursor to an energy source appropriate for initiating polymerization, such as ultraviolet light, heat, a chemical initiator, or combinations thereof. Appropriate polymer precursors, and appropriate energy source for each for initiating polymerization are well understood by those having skill in the art. The ceramic precursor is formed into a porous inorganic gel by aging the mixture at a temperature between room temperature and 60° C. The thus formed porous inorganic gel having a polymer network integrated within the pores of the porous gel may then be impregnated with a bioactive material by soaking the gel in a solution containing the bioactive material, thereby forming the bioactive material as integral to the polymer network. The porous inorganic gel may be formed of a variety of inorganic materials, including but not limited to tetramethylorthosilicate (TMOS), oxide materials, calcium phosphate, hydroxylappetite, calcium carbonate, and mixtures thereof. The pores in the porous inorganic gel are preferably formed as micropores, nanopores, and combinations thereof. Suitable polymers for the present invention include polymers that respond to known changes in their environment, including, but not limited to, temperature and pH. A wide variety of such polymers have been investigated and the selection of a particular polymer will depend on the particular application it is for which it is intended. The various polymers and their characteristic responses to a wide range of external stimuli are well characterized and catalogued. Those having skill in the art will readily recognize appropriate polymers depending on the particular intended use, and will have little difficulty selecting appropriately. For drug release in mammalian bodies controlled by changes in temperature, preferred polymer precursors include, but are not limited to, poly(N-isopropylacrylamide)(PNIPAAm).

The polymer network may further be bonded to target specific modification groups, thereby allowing the polymer network to preferentially bond to bioactive materials characterized by modification groups. Again, appropriate target specific modification groups are well characterized and catalogued, and those having skill in the art will recognize appropriate target modification groups depending on the bioactive materials under consideration. Some such features of bioactive materials that are targets for specific modification groups include, but not limited to metal ions and glucose concentrations.

The porous inorganic gel may also be formed in combination with organic additives, including, without limitation, surfactants, surface modification agents and combinations thereof, to form bicontinuous porous channels within the porous inorganic gel defined by the surfactant termplate. The hybrid porous materials may further be dried and them formed into powders. In applications wherein the hybrid porous materials are going to be introduced into a living organism preferred powders include, but are not limited to, microspheres, nanospheres, and combinations thereof.

In one method for carrying out the present invention, and not meant to be limiting, the ceramic precursor is first formed into a porous inorganic gel, either by the drying method, by a modified sol-gel process, or by a self-assembly method and then calcined to form a hardened inorganic product having a porous network therein. The hardened product may then be grafted to a bonding agent, including but not limited to a silane gel. In this manner, the hardened product can the be mixed with a polymer precursor to form a polymer network bonded to the interior of the pores of the calcined porous inorganic gel and then polymerized to form the hybrid porous material. Alternatively, and not meant to be limiting, the hybrid porous material may also be formed as a coating, a film, a membrane, or combinations thereof by techniques well understood by those having skill in the art including, but not limited to, drip coating and spin coating.

The hybrid porous material be soaked in a solvent to remove any unpolymerized polymer precursor and ungelled ceramic precursor. Suitable solvents are dependant on the specific precursors, and would include, but not be limited to water, alcohols, and combinations thereof. The hybrid porous material may then be dried for later use, or soaked in a bioactive material to form the bioactive material as integral to the polymer network. Bioactive materials, as used herein, include any compounds used for therapeutic or medicinal purposes, or other materials having use in interacting with living things, including but not limited to pharmaceuticals, antibiotics, pesticides, herbicides, insecticides, rodenticides, and the like. The hybrid porous material may further be formed with a bioactive material present, or a bioactive material may later be added to the hybrid porous material by soaking it in the bioactive material to form the bioactive material as integral to the polymer network. This product may be used immediately, or may also be dried for later use.

The subject matter of the present invention is particularly pointed out and distinctly claimed in the concluding portion of this specification. However, both the organization and method of operation, together with further advantages and objects thereof, may best be understood by reference to the following description taken in connection with accompanying drawings wherein like reference characters refer to like elements.

### BRIEF DESCRIPTION OF THE DRAWINGS

FIG. 1 is a schematic drawing of a preferred embodiment of the present invention showing the two layers of the hybrid porous material and bioactive materials therein at different temperatures.

FIG. 2 is a graph of the observed release of Indomethacin at different temperatures in an experiment conducted to demonstrate the advantages of a preferred embodiment of the present invention.

FIG. 3 is a graph comparing the cumulative release of Indomethacin for different pore sizes and water wt. % within the nanogels in an experiment conducted to demonstrate the advantages of a preferred embodiment of the present invention.

### DESCRIPTION OF THE PREFERRED EMBODIMENT(S)

An experiment was conducted to demonstrate a preferred embodiment of the present invention, together with the advantages as compared with polymer drug delivery systems taken in isolation. 75 wt. % water (75% nanogel), 1.4610 g cetylpyridium chloride surfactant (monohydrate form, Aldrich) and 0.3356 g 1-hexanol in 5.0748 g of 0.626% HCl solution were combined and aged overnight. To this thick phase, 3.47 g NIPAAm monomer (recrystallized from hexane) and 0.102 g N,N′-methylene bisacrylamide (NMBA) were added and then shaken until a homogeneous phase formed. Then 10.88 g tetramethylorthosilicate (TMOS, silica precursor) was added. The hydrolysis of TMOS caused the temperature of the solution to rise. After the solution was cooled to room temperature, it was purged with N₂ for 2 hours. Irradiation of this solution with an UV lamp (60 Hz, 2.5 Amps) for 30 minutes caused polymerization, leading to a transparent soft polymer gel. This gel was cured in a 60° C. oven for 3 days to condense the silicate to a hard gel. The thus formed hybrid porous materials, or “nanogels” were also prepared by varying the water content of the initial reagent to form as containing 55 wt. % water and 95 wt. % water.

The nanogels were soaked in a water/EtOH (1:1 volumetric ratio) 5 times to wash out unreacted residues, and dried in air. The dried nanogel was immersed into the saturated solution of indomethacin in EtOH/water (8:2 volumetric ratio) overnight and dried over a period of 3 days at room temperature. The drug loaded nanogel was immersed in 10 mL phosphate buffer (pH=7.4, 10 mM). Solutions containing the drug loaded nanogel were aged in an Environ Shaker for stepwise temperature changes between 25° C. and 40° C. The indomethacin concentration of the solution was measured using an UV-Vis spectrophotometer (257 nm) at different time intervals. After each measurement, 10 mL of PBS buffer was replaced. In all the experiments, the samples were roughly disk like and approximately 5 mm in the lateral dimension and 2 mm in thickness.

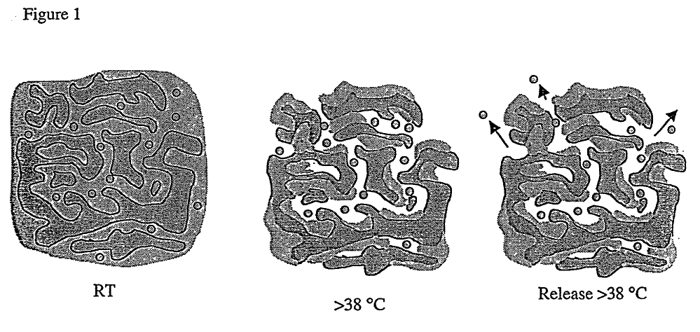

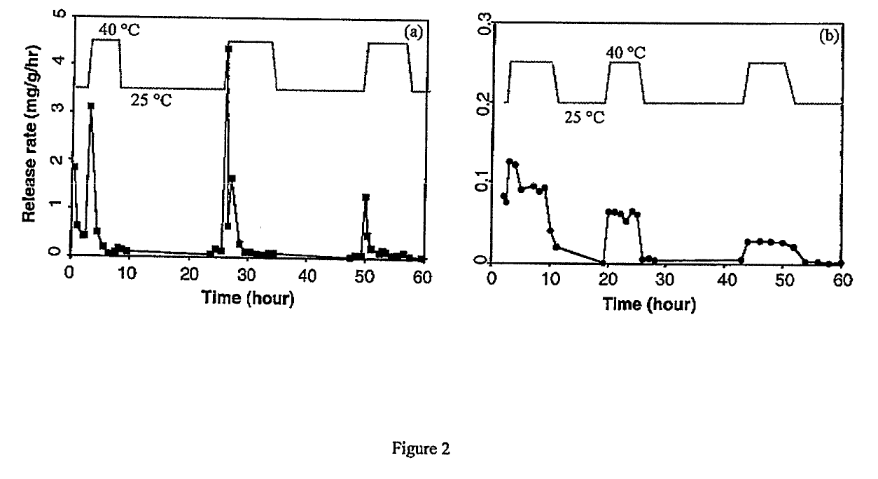

The thus formed nanogels are represented schematically in FIG. 1 where the iorganic phase is represented as 1., the polymer phase as 2. and the Indomethacin as 3. at varying temperatures. At room temperature, the release was quenched. Release rates were then measured as the temperature was cycled between 40° C. and 25° C. and compared to a Poly N-isopropylacrylamide system loaded with Indomethacin. As shown in FIG. 2, the release rate for both the hybrid porous material (nanogels) and the pure Poly N-isopropylacrylamide are greatly elevated when the temperature is elevated to 40° C. However, the pure Poly N-isopropylacrylamide system reacts with a large spike in its release rate, and then and then drop precipitously while the temperature is still maintained at 40° C. In contrast, the hybrid porous material maintains an essentially steady state release at 40° C., and maintains that release rate until the temperature is reduced to 25° C.

In the experiments conducted to demonstrate a preferred embodiment of the present invention, nanodiffusion in the nanoporous channels, is the controlling diffusion mechanism. This is distinct from the in the surface area or the diffusion in the gel phase as reported in prior art systems. When the temperature of the drug is increased to 40° C., there is an increased release from the gel phase, but the drug is not released to the medium. The drug must first diffuse through the nanochannels. The effective diffusion constant in the nanochannels is smaller than the bulk diffusion. For example, for 55 wt. % nanogels, the pore dimension is about 8 nm. Assuming a drug size of about 2 nm, the effective diffusion constant is only 22% of that of the bulk value. This mechanism slows down the diffusion at the onset of the temperature change even though a large concentration of the drug is delivered to the pores from the gel phase. This reduced diffusion rate also helps maintain a more or less constant drug level in the pore channels. Therefore the nanodiffusion mechanism can help avoid the spike-like release profile, and maintain a more uniform release rate.

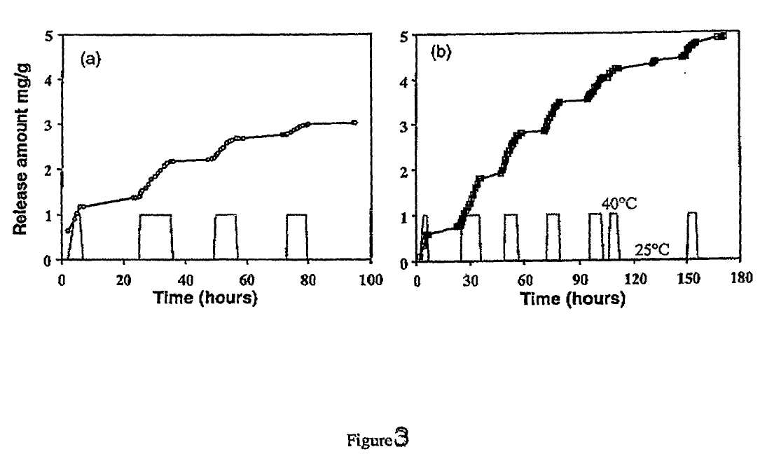

Reducing the water content from 95 wt. % to 55 wt. % can reduce the pore dimensions from about 30 nm to about 8 nm in the hybrid porous materials. FIG. 3 compares the cumulative amount of indomethcin released as a function of time when the temperature is changed between 25° C. and 40° C. for a 95 wt. % nanogel and a 55 wt. % nanogel. In the 95 wt. % nanogel, although the positive on and off mechanism is clearly observed (a positive on and off mechanism is characterized by being “on”, or allowing release, in response to an elevated temperature, and “off” in response to a lowered temperature) when the temperature is changed, by the third cycle (after 70 hours), the release rate was reduced by several folds. However, by reducing the amount of water in the 55 wt. % nanogel, the release is extended to a much longer period of time. The release rate did not decrease significantly after six cycles (a duration of more than 150 hours).

### Closure

While a preferred embodiment of the present invention has been shown and described, it will be apparent to those skilled in the art that many changes and modifications may be made without departing from the invention in its broader aspects. The appended claims are therefore intended to cover all such changes and modifications as fall within the true spirit and scope of the invention.

We claim:

1. A hybrid porous material for controlled release of a bioactive material comprising:
a) a porous inorganic material having
b) a polymer network integrated within the pores of the porous inorganic material.

2. The hybrid porous material of claim 1 further comprising a bioactive material integral to said polymer network.

3. The hybrid porous material of claim 1 wherein the porous inorganic material is selected from the group consisting of an oxide material, calcium phosphate, hydroxylappetite, calcium carbonate, and mixtures thereof.

4. The hybrid porous material of claim 1 wherein pores within the porous inorganic material are selected from the group consisting of micropores, nanopores, and combinations thereof.

5. The hybrid porous material of claim 1 wherein the polymer network is selected as poly (N-isopropylacrylamide).

6. The hybrid porous material of claim 1 wherein the polymer network further comprises target specific modification groups.

7. The hybrid porous material of claim 1 wherein the inorganic material further comprises organic additives, surfactants, surface modification agents and combinations thereof.

8. The hybrid porous material of claim 1 wherein the hybrid porous material is in the form of a powder, a microsphere, or combinations thereof.

9. The hybrid porous material of claim 1 wherein the hybrid porous material is in the form of a coating, a film, a membrane, or combinations thereof.

10. A method for forming a hybrid porous material for controlled release of a bioactive material comprising the steps of:
a) forming a mixture of a polymer precursor, a cross linking agent, an initiator, an alcohol, a ceramic precursor, water and an acid,
b) polymerizing the polymer precursor to form a polymer network, and
c) forming the ceramic precursor into a porous inorganic gel, thereby forming a porous inorganic gel having a polymer network integrated within the pores of the porous gel.

11. The method of claim 10 wherein the step of polymerizing the polymer precursor is accomplished by exposing the polymer precursor to ultraviolet light, heat, or combinations thereof.

12. The method of claim 10 wherein the step of forming the ceramic precursor into a porous inorganic gel is accomplished by aging the mixture at a temperature between room temperature and 60° C.

13. The method of claim 10 further comprising the step of soaking the porous inorganic gel in a bioactive material, thereby forming the bioactive material as integral to the polymer network.

14. The method of claim 10 further comprising the step of providing the porous inorganic gel as selected from the group consisting of an oxide material, calcium phosphate, hydroxylappetite, calcium carbonate, and mixtures thereof.

15. The method of claim 10 further comprising the step of providing the pores in the porous inorganic gel as selected from the group consisting of micropores, nanopores, and combinations thereof.

16. The method of claim 10 further comprising the step of providing the polymer precursor as poly(N-isopropylacrylamide).

17. The method of claim 10 further comprising the step of providing the polymer network bonded to target specific modification groups.

18. The method of claim 10 further comprising the step of providing the porous inorganic gel in combination with organic additives, surfactants, surface modification agents and combinations thereof.

19. The method of claim 10 further comprising the step of grinding the porous inorganic gel having a polymer network integrated within the pores of the porous inorganic gel to form a powder, a microsphere, or combinations thereof.

20. The method of claim 10 further comprising the step of forming the porous inorganic gel having a polymer network integrated within the pores of the porous inorganic gel as a coating, a film, a membrane, or combinations thereof.

21. The method of claim 10 further comprising the step of soaking the porous inorganic gel having a polymer network integrated within the pores of the porous inorganic gel in a solvent to remove any unpolymerized polymer precursor and ungelled ceramic precursor.

22. The method of claim 10 further comprising the step of soaking the porous inorganic gel having a polymer network integrated within the pores of the porous inorganic gel in a bioactive material to form the bioactive material as integral to the polymer network.
